(19) United States
(12) Patent Application Publication
(10) Pub. No.: US 2013/0137111 A1
(43) Pub. Date: May 30, 2013

(54) DETECTION METHOD OF NOVEL RET FUSION

(71) Applicants: Nobuaki Shindo, Tokyo (JP); Kouichi Nishimura, Tokyo (JP); Yorikata Sano, Tokyo (JP); Makoto Asaumi, Tokyo (JP); Aya Yamanaka, Tokyo (JP); Takashi Futami, Tokyo (JP); Tatsuya Kawase, Tokyo (JP)

(72) Inventors: Nobuaki Shindo, Tokyo (JP); Kouichi Nishimura, Tokyo (JP); Yorikata Sano, Tokyo (JP); Makoto Asaumi, Tokyo (JP); Aya Yamanaka, Tokyo (JP); Takashi Futami, Tokyo (JP); Tatsuya Kawase, Tokyo (JP)

(73) Assignee: Astellas Pharma Inc., Tokyo (JP)

(21) Appl. No.: 13/812,356

(22) PCT Filed: Jul. 22, 2011
(86) PCT No.: PCT/JP11/66661

§ 371 Date: Jan. 25, 2013

(30) Foreign Application Priority Data
Jul. 26, 2010 (JP) 2010-166674

(51) Int. Cl. C12Q 1/68

(52) U.S. Cl. CPC C12Q 1/6886
USPC 435/6.12

(57) ABSTRACT

It was found that a new fusion gene of a portion of the KIF5B gene and a portion of the RET gene, which is present in a portion of cancer patients, is a gene responsible for cancer. Based on this finding, detection methods of a polynucleotide as the gene and a polypeptide encoded by the polynucleotide were established. The detection methods involve detecting a fusion gene of a portion of the KIF5B gene and a portion of the RET gene, or a fusion protein encoded by such a fusion gene. The primer sets and the detection kits comprise sense primers designed from a portion encoding KIF5B and antisense primers designed from a portion encoding RET.

### TECHNICAL FIELD

The present invention relates to a detection method of a novel fusion gene that has an RET kinase domain or a fusion protein that is encoded by the fusion gene.

### BACKGROUND ART

A rearranged during transformation (RET) gene is mapped near the centromere of the long arm of chromosome 10 and consists of 21 exons. A protein encoded by this gene is receptor-type tyrosine kinase. The RET gene has a transmembrane domain in the central portion, a tyrosine kinase domain on a carboxyl terminal and an extracellular domain on an amino terminal. It is known that there are three types of proteins including 1072 amino acids (RET 9), 1106 amino acids (RET 43) and 1114 amino acids (RET 51) due to the different splicing pattern in the carboxyl terminal (Non-Patent Document 1). By forming a dimer via a ligand/GFR complex, RET phosphorylates and activates its own tyrosine (Non-Patent Document 1).

It is reported that the RET gene is fused with other genes (H4, ELE1 and the like) due to intrachromosomal inversion or interchromosomal translocation, whereby fusion tyrosine kinase RET/PTC having an transforming activity is expressed in papillary thyroid cancer (Non-Patent Document 2). The fusion is caused on the amino terminal in the tyrosine kinase domain of RET, and RET loses the extracellular domain and the transmembrane domain and forms a fusion protein with a fusion partner. Since many of the fusion partners have a domain for forming a complex, the fusion protein itself is also considered to form a complex, and it is considered that the formation of the complex causes the constitutive autophosphorylation of tyrosine kinase activity of RET, and intracellular signaling is aberrantly activated, which induces oncogenic transformation and cell growth (Non-Patent Document 2). Actually, when the RET/PTC fusion gene is transduced into normal mouse NIH3T3 cells, the cells are transformed into cells like cancer cells, and an RET kinase inhibitor can inhibit the cell growth of a thyroid cancer cell line expressing the RET/PTC fusion gene. These facts suggest that the RET/PTC fused is likely to be a target molecule in treating thyroid cancer (Non-Patent Documents 3 to 5). So far, it has been reported that 10 or more kinds of RET/PTC fusion genes are found in thyroid cancer, but there is no report showing that the RET fusion genes are found in lung cancer.

A kinesin family protein 5B (KIF5B) gene is mapped in the short arm of chromosome 10 and consists of 26 exons. It is known that KIF5B is one of the molecules constituting a motor protein complex involved in microtubule transport in a cell, and it is involved in the intracellular transport of materials with a characteristic of moving along the microtubule (Non-Patent Document 6).

So far, it has been reported that a fusion protein of KIF5B and receptor-type tyrosine kinase PDGFRα is expressed in a patient with hypereosinophilia (Non-Patent Document 7). In addition, it is reported that a fusion protein of KIF5B and Anaplastic Lymphoma Kinase as receptor-type tyrosine kinase is found in non-small cell lung cancer (Non-Patent Document 8), but there is no report showing that KIF5B is fused with RET.

### RELATED ART

### Non-Patent Document

[Non-Patent Document 1] TRENDS in Genetics, (UK), 2006, Vol. 22, p. 627-636

[Non-Patent Document 2] European journal of endoclinology, (UK), 2006, Vol. 155, p. 645-653

[Non-Patent Document 3] CANCER RESEARCH, (USA), 2002, Vol. 62, p. 7284-7290

[Non-Patent Document 4] The journal of Clinical Endocrinology & Metabolism, (USA), 2006, Vol. 91, p. 4070-4076

[Non-Patent Document 5] THE JOURNAL OF BIOLOGICAL CHEMISTRY, (USA), 2007, Vol. 282, p. 29230-29340

[Non-Patent Document 6] NATURE REVIEWS Molecular cell Biology, (UK), 2009, Vol. 10, p. 877-884

[Non-Patent Document 7] Leukemia, (UK), 2006, Vol. 20, p. 827-832

[Non-Patent Document 8] CLINICAL CANCER RESEARCH, (USA), 2009, Vol. 15, p. 3143-3149

### DISCLOSURE OF INVENTION

### Problem to be Solved by the Invention

An object of the present invention is to define a polynucleotide as a novel gene responsible for cancer, thereby providing a detection method of the polynucleotide or a polypeptide encoded by the polynucleotide and a kit as well as a primer set for the method.

### Means for Solving the Problems

The present inventors isolated and identified novel fusion genes in which a portion of KIF5B gene was fused with a portion of RET gene as kinase from specimens obtained from patients with lung cancer (Example 1). As a result, they found that those fusion genes are present in the specimen from the patient with lung cancer (Example 2), and the fusion genes have tumorigenicity and are genes responsible for cancer (Examples 3, 4 and 6). Based on the knowledge, the present inventors established detection methods of those fusion genes and fusion proteins encoded by the fusion genes (Examples 2 and 5), and provided kits and primer sets for the method. Detection of those fusion genes or the fusion proteins encoded by the fusion genes makes it possible to screen cancer patients to be subjects who will receive drug therapy using RET inhibitors.

That is, the present invention relates to the following [1] to [9].

[1] A detection method of a fusion gene of Kinesin family protein 5B (KIF5B) gene and rearranged during transformation (RET) gene or a fusion protein encoded by the fusion gene, comprising:

a step of detecting the presence of a polynucleotide encoding the following polypeptide or presence of the polypeptide in a sample obtained from a test subject:

a polypeptide that comprises an amino acid sequence having 90% or higher identities with the amino acid sequence represented by SEQ ID NO: 2 or 4 and has tumorigenicity.

[2] The method according to [1],

wherein the polypeptide is a polypeptide that comprises the amino acid sequence represented by SEQ ID NO: 2 or 4 and has tumorigenicity, or a polypeptide that comprises an amino acid sequence formed by deletion, substitution and/or insertion of 1 to 10 amino acids in the amino acid sequence represented by SEQ ID NO: 2 or 4 and has tumorigenicity.

[3] The method according to [1] or [2],

wherein the polypeptide is a polypeptide consisting of the amino acid sequence represented by SEQ ID NO: 2 or 4.

[4] A kit for detecting a fusion gene of KIF5B gene and RET gene, comprising:

sense and antisense primers designed so as to be able to specifically amplify a polynucleotide encoding the following polypeptide:

a polypeptide that comprises an amino acid sequence having 90% or higher identities with the amino acid sequence represented by SEQ ID NO: 2 or 4 and has tumorigenicity.

[5] The kit according to [4],

wherein the polypeptide is a polypeptide that comprises the amino acid sequence represented by SEQ ID NO: 2 or 4 and has tumorigenicity, or a polypeptide that comprises an amino acid sequence formed by deletion, substitution and/or insertion of 1 to 10 amino acids in the amino acid sequence represented by SEQ ID NO: 2 or 4 and has tumorigenicity.

[6] The kit according to [4] or [5],

wherein the polypeptide is a polypeptide consisting of the amino acid sequence represented by SEQ ID NO: 2 or 4.

[7] A primer set for detecting a fusion gene of KIF5B gene and RET gene, comprising:

a sense primer designed from a portion encoding the KIF5B gene; and

an antisense primer designed from a portion encoding the RET gene,

wherein the antisense primer consists of a nucleic acid molecule that is hybridized with a polynucleotide encoding the polypeptide according to any one of [1] to [3] under a stringent condition, and

the sense primer consists of a nucleic acid molecule that is hybridized with a complementary strand of the polynucleotide under a stringent condition.

[8] A primer set of

a sense primer consisting of an oligonucleotide with at least any 16 consecutive bases located at base numbers 1 and 1725 of SEQ ID NO: 1 and

an antisense primer consisting of an oligonucleotide complementary to an oligonucleotide with at least any 16 consecutive bases located at base numbers 1726 and 2808 of SEQ ID NO: 1, or a primer set consisting of complementary strands thereof,

wherein a size of an amplification product amplified by the sense primer and the antisense primer is 1 kb or less.

[9] A primer set of

a sense primer consisting of an oligonucleotide with at least any 16 consecutive bases located at base numbers 1 and 1725 of SEQ ID NO: 3 and

an antisense primer consisting of an oligonucleotide complementary to an oligonucleotide with at least any 16 consecutive bases located at base numbers 1726 and 2934 of SEQ ID NO: 3, or a primer set consisting of complementary strands thereof,

wherein a size of an amplification product amplified by the sense primer and the antisense primer is 1 kb or less.

The present invention also relates to a detection method of the presence of cancer (particularly lung cancer) positive for a fusion gene having RET kinase domain, including:

a step of detecting the presence of a polynucleotide that encodes polypeptides according to the following sections (1) to (3) in a sample obtained from a test subject,

(1) a polypeptide that comprises the amino acid sequence represented by SEQ ID NO: 2 or 4 and has tumorigenicity, or a polypeptide that comprises an amino acid sequence formed by deletion, substitution and/or insertion of 1 to 10 amino acids in the amino acid sequence represented by SEQ ID NO: 2 or 4 and has tumorigenicity,

(2) a polypeptide that comprises an amino acid sequence having 90% or higher identities with the amino acid sequence represented by SEQ ID NO: 2 or 4 and has tumorigenicity, or

(3) a polypeptide that consists of the amino acid sequence represented by SEQ ID NO: 2 or 4.

### Effects of the Invention

The detection methods of the present invention can be used as a method of detecting cancer (particularly lung cancer) positive for a fusion gene of KIF5B gene and RET gene. The detection methods of the present invention can also be used as a method of detecting chromosomal rearrangement. In addition, according to the detection methods of the present invention, it is possible to determine whether or not a patient is a test subject who will be administered with RET inhibitors, and as a result, it is possible to provide tailor-made treatment in which RET inhibitor therapy is expected to be effective. The detection kits and primer sets of the present invention can be used for the detection method of the present invention.

### EMBODIMENTS FOR CARRYING OUT THE INVENTION

<Detection Method of the Present Invention>

The detection method of the present invention includes (1) a detection method of a fusion gene, and (2) a detection method of a fusion protein encoded by the fusion gene.

(1) Detection Method of Fusion Gene

The detection method of a fusion gene of the present invention includes a step of detecting the presence of polynucleotides of the present specification in a sample obtained from a test subject.

As the sample obtained from a test subject, substances collected from a test subject (samples isolated from a living body), specifically, any types of body fluid (preferably blood) collected, alveolar and bronchial washings, samples having undergone biopsy, and phlegm samples are used. Preferably, a biopsy sample or a phlegm sample from an affected area in the lung of a test subject is used. From the sample, genome DNA can be extracted and used. In addition, transcripts thereof (products produced as a result of transcription and translation of a genome; for example, mRNA, cDNA and proteins) can be used. Particularly, it is preferable to prepare and use mRNA or cDNA.

In the “step of detecting the presence of polynucleotides of the present specification” in the detection method of a fusion gene, the polynucleotides of the present specification to be detected (hereinafter, called polynucleotides to be detected) refer to polynucleotides that encode polypeptides as fusion proteins of a portion of KIF5B and a portion of RET having RET kinase domain. As the polynucleotides to be detected, polynucleotides encoding polypeptides according to the following sections (i) to (iii) are particularly preferable,

(i) a polypeptide that comprises the amino acid sequence represented by SEQ ID NO: 2 or 4 and has tumorigenicity, or a polypeptide that comprises an amino acid sequence formed by deletion, substitution and/or insertion of 1 to 10 amino acids in the amino acid sequence represented by SEQ ID NO: 2 or 4 and has tumorigenicity (hereinafter, called a functionally equivalent variant),

(ii) a polypeptide that comprises an amino acid sequence having 90% or higher identities with the amino acid sequence represented by SEQ ID NO: 2 or 4 and has tumorigenicity (hereinafter, called a homologous polypeptide), and

(iii) a polypeptide that consists of the amino acid sequence represented by SEQ ID NO: 2 or 4.

The amino acid sequence represented by SEQ ID NO: 2 is a sequence encoded by the base sequence represented by SEQ ID NO: 1. The amino acid sequence represented by SEQ ID NO: 4 is a sequence encoded by the base sequence represented by SEQ ID NO: 3. A protein which is a polypeptide consisting of the amino acids represented by SEQ ID NO: 2 is called a “KIF5B-RET fusion polypeptide v1S”, and a protein which is a polypeptide consisting of the amino acids represented by SEQ ID NO: 4 is called a “KIF5B-RET fusion polypeptide v1L”. The “KIF5B-RET fusion polypeptide v1S” and the “KIF5B-RET fusion polypeptide v1L” are collectively called a “KIF5B-RET fusion polypeptide v1”. A gene encoding the KIF5B-RET fusion polypeptide v1S is called a “KIF5B-RET fusion polynucleotide v1S”, and a gene encoding the KIF5B-RET fusion polypeptide v1L is called a “KIF5B-RET fusion polynucleotide v1L”. A gene encoding the KIF5B-RET fusion polypeptide v1 is called a “KIF5B-RET fusion polynucleotide v1”.

As a “functionally equivalent variant”, a “polypeptide that comprises an amino acid sequence formed by deletion, substitution and/or insertion of 1 to 10 amino acids, preferably 1 to several amino acids, more preferably 1 to 7 amino acids, and most preferably 1 to 5 amino acids in the amino acid sequence represented by SEQ ID NO: 2 or 4 and has tumorigenicity” is preferable, and a “polypeptide that comprises the amino acid sequence represented by SEQ ID NO: 2 or 4 and has tumorigenicity” is particularly preferable.

A “homologous polypeptide” is a “polypeptide that comprises an amino acid sequence having 90% or higher identities with the amino acid sequence represented by SEQ ID NO: 2 or 4 and has tumorigenicity”. As the homologous polypeptide, a polypeptide that comprises an amino acid sequence preferably having 95% or higher identities and more preferably having 98% or higher identities is preferable.

In addition, the “identities” in the present specification refers to Identities which is a value obtained using parameters prepared as default parameters by the search using NEEDLE program (J Mol Biol 1970; 48: 443-453). The parameters are as follows.

Gap penalty=10

Extend penalty=0.5

Matrix=EBLOSUM62

Whether a certain polypeptide has “tumorigenicity” is confirmed by the method of Example 4. Specifically, a host (3T3 fibroblast cell) into which a plasmid expressing the polypeptide has been transduced is subcutaneously inoculated into nude mice, and whether a tumor is formed is determined to confirm tumorigenicity.

As the polynucleotide to be detected in the detection method of the present invention, a polynucleotide encoding “a polypeptide that comprises the amino acid sequence represented by SEQ ID NO: 2 or 4 and has tumorigenicity” is preferable, and a polynucleotide encoding “a polypeptide that consists of the amino acid sequence represented by SEQ ID NO: 2 or 4” is most preferable.

The “step of detecting the presence of a polynucleotide” in the detection method of a polynucleotide of the present invention is performed by detecting the presence of a polynucleotide to be detected (genome sequence including a fusion point) in a genome of a sample obtained from a test subject, or by preparing a transcript (for example, mRNA or cDNA) of genome DNA extracted from a sample obtained from a test subject and detecting the presence of mRNA or cDNA corresponding to the polynucleotide to be detected.

Genome DNA can be extracted by known methods, and the extraction can be easily performed using a commercially available DNA extraction kit.

The step of detection can be performed according to known gene analysis methods (for example, known methods that are commonly used as gene detection methods such as PCR, LCR (Ligase chain reaction), SDA (Strand displacement amplification), NASBA (Nucleic acid sequence-based amplification), ICAN (Isothermal and chimeric primer-initiated amplification of nucleic acids), LAMP (Loop-mediated isothermal amplification) method, TMA (Gen-Probe's TMA system) method, in situ hybridization method, and microarrays). For example, a hybridization technology in which a nucleic acid hybridized with a polynucleotide to be detected is used as a probe, a gene amplification technology in which DNA hybridized with a polynucleotide to be detected is used as a primer, or the like is used.

Specifically, the detection is performed using nucleic acids derived from a sample obtained from a test subject, for example, mRNA and the like. The amount of mRNA is measured by a method of gene amplification reaction by using primers that are designed so as to be able to specifically amplify the sequence of a polynucleotide to be detected. The primers used in the detection method of the present invention, or the primers included in the detection kit are not particularly limited as long as the primers can specifically amplify the sequence of a polynucleotide to be detected, and designed based on the base sequence of a polynucleotide to be detected. Primers used in the PCR amplification monitor method can be designed using primer design software (for example, Primer Express manufactured by PE Biosystems) and the like. In addition, since the larger the size of a PCR product is, the more the amplification efficiency worsens, it is appropriate for a sense primer and an antisense primer to be designed such that the size of amplification products obtained when mRNA or cDNA is amplified becomes 1 kb or less.

More specifically, a sense primer (5′-primer) is designed from a portion encoding KIF5B, and an antisense primer (3′-primer) is designed from a portion encoding RET. It is preferable to use the primer included in the detection kit of the present invention, and it is more preferable to use the primer that is most suitably included in the detection kit. In the PCR amplification monitor method, it is also possible to design multiplex PCR for detecting all fusion polynucleotides in a single reaction liquid, by mixing the above sense primers corresponding to respective genes. By the method suitable for each amplification technology, it is possible to confirm whether or not a target gene (whole gene or a specific portion thereof) has been amplified. For example, in the PCR method, PCR products are analyzed by agarose gel electrophoresis and subjected to ethidium bromide staining and the like, whereby it is possible to confirm whether or not amplified fragments having a target size have been obtained. When the amplified fragments having a target size have been obtained, this indicates that a polynucleotide to be detected is present in the sample obtained from a test subject. The presence of a polynucleotide to be detected can be detected in this manner.

The detection method of a fusion gene of the present invention preferably includes a step of detecting the presence of a specific polynucleotide in a sample obtained from a test subject by a gene amplification reaction and a step of detecting whether or not amplified fragments having a target size have been obtained.

The detection using a hybridization technology is performed using, for example, northern hybridization, dot blotting method, DNA microarray method, and RNA protection method. As probes used for hybridization, it is possible to use a probe which comprises sequences consisting of 16 bases respectively upstream and downstream of the fusion point as a center of a nucleic acid molecule consisting of at least 32 consecutive bases hybridizing with a polynucleotide to be detected or with a complementary strand thereof in a stringent condition (preferably in a more stringent condition), or comprises complementary strands thereof.

The detection using an in situ hybridization technology can be performed according to a known FISH method (fusion assay). In addition, the detection can be performed by a fusion assay which is a combination of chromogenic in situ hybridization (CISH) method and silver in situ hybridization (SISH) method.

Moreover, the “fusion point” in the present specification refers to a point where a portion derived from the respective genes of KIF5B is fused with a portion derived from the RET gene.

Further, the “stringent condition” in the present specification refer to “5× SSPE, 5× Denhardt's solution, 0.5% SDS, 50% formamide, 200 μg/mL salmon sperm DNA, 42° C. overnight” as conditions for hybridization, and “0.5×SSC, 0.1% SDS, 42° C.” as conditions for washing. The “more stringent condition” refer to “5× SSPE, 5× Denhardt's solution, 0.5% SDS, 50% formamide, 200 μg/mL salmon sperm DNA, 42° C. overnight” as conditions for hybridization, and “0.2×SSC, 0.1% SDS, 65° C.” as conditions for washing.

It is also possible to use a gene amplification technology such as RT-PCR. In the RT-PCR method, the PCR amplification monitor (real time PCR) method (Genome Res., 6(10), 986, 1996) is performed during the process of gene amplification, whereby the presence of a polynucleotide to be detected can be more quantitatively analyzed. In the PCR amplification monitor method, for example, ABI PRISM7900 (manufactured by PE Biosystems) can be used. Real time PCR is a known method, and can be simply performed using commercially available instruments and kits for this method.

(2) Detection Method of Fusion Protein Encoded by the Above Fusion Gene

The detection method of a fusion protein of the present invention includes a step of detecting the presence of a specific polypeptide in a sample obtained from a test subject, that is, a polypeptide encoded by a polynucleotide to be detected (hereinafter, called a polypeptide to be detected).

Such a detection step can be performed by immunoassay method or enzyme activity assay method that is conducted by preparing a solubilized liquid derived from a sample obtained from a test subject (for example, a cancer tissue or cells obtained from a test subject) and combining a polypeptide to be detected contained in the liquid with an anti-KIF5B antibody and an anti-RET antibody. Preferably, it is possible to use techniques using a monoclonal antibody or a polyclonal antibody specific to a polypeptide to be detected, such as enzymatic immunoassay method, double antibodies sandwich ELISA method, fluorescence immunoassay method, radioimmunoassay method, and western blotting method.

When the polynucleotide to be detected or polypeptide to be detected in the detection method of the present invention is detected from a sample obtained from a test subject, the test subject is a subject (patient) who has cancer with the polynucleotide positive and is to be provided with treatment using RET inhibitors.

<Detection Kit of the Present Invention and Primer Set of the Present Invention>

The detection kit of the present invention comprises at least sense and antisense primers that are designed so as to be able to specifically amplify a polynucleotide to be detected in the detection method of the present invention. The sense and antisense primer set is a set of polynucleotides functioning as primers for amplifying a polynucleotide to be detected.

The “fusion gene of KIF5B gene and RET gene” refers to a gene in which a portion of the KIF5B gene is fused with a portion of the RET gene.

The primer set of the present invention comprises (1) a primer set which comprises a sense primer designed from a portion encoding KIF5B and an antisense primer designed from a portion encoding RET and is for detecting a fusion gene of KIF5B gene and RET gene, wherein the antisense primer consists of a nucleic acid molecule (preferably a nucleic acid molecule consisting of at least 16 bases) hybridizing with a “polynucleotide to be detected” under a stringent condition (preferably under a more stringent condition), and the sense primer consists of a nucleic acid molecule (preferably a nucleic acid molecule consisting of at least 16 bases) hybridizing with a complementary strand of the “polynucleotide to be detected” under a stringent condition (preferably under a more stringent condition).

In addition, as a more specific embodiment of the primer set (1), the following primer sets (2) and (3) are included in the primer set of the present invention.

(2) A primer set of a sense primer that consists of an oligonucleotide consisting of at least any 16 consecutive bases between base numbers 1 and 1725 of SEQ ID NO: 1 and an antisense primer that consists of an oligonucleotide complementary to an oligonucleotide consisting of at least any 16 consecutive bases between base numbers 1726 and 2808 of SEQ ID NO: 1

(3) A primer set of a sense primer that consists of an oligonucleotide consisting of at least any 16 consecutive bases between base numbers 1 and 1725 of SEQ ID NO: 3 and an antisense primer that consists of an oligonucleotide complementary to an oligonucleotide consisting of at least any 16 consecutive bases between base numbers 1726 and 2934 of SEQ ID NO: 3

In these primer sets (1) to (3), an interval between the positions where the sense primer and the antisense primer are selected is preferably 1 kb or less, or the size of an amplification product amplified by the sense primer and the antisense primer is preferably 1 kb or less.

In addition, the primer of the present invention has a strand length consisting of 15 to 40 bases in general, preferably consisting of 16 to 24 bases, more preferably consisting of 18 to 24 bases, and particularly preferably consisting of 20 to 24 bases.

In the detection method of the present invention, the primer set of the present invention can be used for amplifying and detecting a polynucleotide to be detected. Moreover, though not particularly limited, the respective primers included in the primer set of the present invention can be prepared by, for example, chemical synthesis.

### EXAMPLES

Hereinafter, the present invention will be described in detail based on examples, but the present invention is not limited to the examples. In addition, unless otherwise specified, the present invention can be embodied according to known methods. Moreover, when commercially available reagents or kits are used, the present invention can be embodied according to the instruction of the commercially available products.

### Example 1

### Isolation of KIF5B-RET Fusion Polynucleotide v1

A reverse transcription reaction was performed on Lg 165 specimen RNA as RNA derived from a lung cancer tissue of a patient with non-small cell lung cancer (Asterand USA) by using reverse transcriptase (SuperScript III, Life Technologies) and oligo(dT) primer (Oligo(dT) 20 primer, Life Technologies), according to the protocol of the kit, thereby synthesizing cDNA.

Thereafter, by using primers of SEQ ID NOS: 5 and 6 including a recognition sequence for a restriction enzyme NotI, the cDNA obtained as above as a template, and a DNA polymerase (PrimeSTAR HS DNA Polymerase; TAKARA BIO INC.), PCR (98° C. for 10 seconds, and then 60° C. for 15 seconds and 68° C. for 3.5 minutes, 35 cycles) was performed, followed by electrophoresis. As a result, it was found that about 3 kb of PCR products were obtained. The PCR products were digested with NotI and cloned into an NotI site present in a multicloning site of an expression vector (pTracer-CMV-bsd; Life Technologies), thereby establishing an expression plasmid. For insertion, the sequence was determined by dideoxy sequencing (BigDye Terminator v3.1 Cycle Sequencing Kit; Life Technologies). As a result, the presence of a transcript (KIF5B-RETv1S) (SEQ ID NO: 1) in which a portion from the N-terminal to exon 15 of a coding sequence (hereinafter, referred to as CDS) of KIF5B (NM_{—}004521) registered in NCBI was fused with a portion from exon 12 of CDS to the C-terminal of CDS of a short form (NM_{—}020630) of RET was clarified. A polypeptide encoded by SEQ ID NO: 1 is shown in SEQ ID NO: 2.

In addition, PCR was performed in the same manner as above by using primers of SEQ ID NOS: 5 and 7 including the NotI sequence, followed by cloning. As a result, the presence of a transcript (KIF5B-RETv1L) (SEQ ID NO: 3) in which a portion from the N-terminal to exon 15 of CDS of KIF5B registered in NCBI was fused with a portion from exon 12 of CDS to the C-terminal of CDS of a long form (NM_{—}020975) of RET was observed. A polypeptide encoded by SEQ ID NO: 3 is shown in SEQ ID NO: 4.

Moreover, in order to express a full length ORF of KIF5B-RETv1S and KIF5B-RETv1L as a protein in which a FLAG tag is added to the N-terminal, by using primers of SEQ ID NOS: 8 and 6 into which the NotI site and the FLAG sequence were introduced and primers of SEQ ID NOS: 8 and 7, and using PCR products including the full length ORF of KIF5B-RETv1S and KIF5B-RETv1L as templates respectively, PCR was performed in the same conditions as above. The products were cloned into the NotI site present in the multicloning site of an expression vector (pTracer-CMV-bsd; Life Technologies), thereby establishing expression plasmids (FLAG-KIF5B-RETv1S/pTracer-CMV-bsd and FLAG-KIF5B-RETv1L/pTracer-CMV-bsd).

### Example 2

### Detection of KIF5B-RET Fusion Polynucleotide v1

By using cDNA 181 samples derived from clinical specimens of non-small cell lung cancer (Asterand USA) as substrates, oligonucleotides consisting of the base sequences represented by SEQ ID NOS: 9 and 10 as a primer set, a quantitative PCR kit (Power SYBR Green PCR Master Mix; Life Technologies), and Applied Biosystems 7900HT system, quantitative PCR (95° C. for 10 minutes, and then 95° C. for 15 seconds and 59° C. for 60 seconds, 45 cycles) was performed to measure the gene expression level. As a result, amplification was confirmed only in the above sample Lg 165. The results show that by the above method, it is possible to detect the presence of the KIF5B-RET polynucleotide v1 in a sample derived from a clinical specimen of non-small cell lung cancer, and to screen a KIF5B-RET polynucleotide v1 positive patient.

### Example 3

### Examination of Growth Factor-Independent Growth Acceleration Effect of KIF5B-RET Fusion Polynucleotide v1

2 μg of the above FLAG-KIF5B-RETv1S/pTracer-CMV-bsd or FLAG-KIF5B-RETv1L/pTracer-CMV-bsd was introduced respectively into BA/F3 cells by using transfection reagent (Cell Line Nucleofector Kit V; Lonza) by electroporation (Nucleofector; Lonza), and cultured in RPMI1640 medium (Invitrogen) obtained by adding 10% bovine serum (Invitrogen) and 1 ng/mL of mouse recombinant IL-3 (R&D systems). Two days after the introduction, the medium was replaced with medium obtained by adding 10 μg/mL of Blastcidin (Invitrogen) to the above medium, and then the cells were cultured continuously for two more weeks. As a result, BA/F3 cells stably expressing KIF5B-RETv1S in which FLAG tag was added to the N-terminal and BA/F3 cells stably expressing KIF5B-RETv1L in which FLAG tag was added to the N-terminal (named FLAG-KIF5B-RETv1S expression/BA/F3 cells and FLAG-KIF5B-RETv1L expression/BA/F3 cells respectively) were obtained.

The growth potential of the FLAG-KIF5B-RETv1S expression/BA/F3 cells and FLAG-KIF5B-RETv1L expression/BA/F3 cells was examined. The FLAG-KIF5B-RETv1S expression/BA/F3 cells, FLAG-KIF5B-RETv1L expression/BA/F3 cells, and BA/F3 cells as the parent strain were seeded in a 96-well plate at 2×10³ cells per well and cultured for 8 days in the absence of growth factor IL-3, and cell counting reagent (CELLTITER-Gio™ Luminescent Cell Viability Assay; Promega Corporation) was added thereto, followed by stirring for 20 minutes. Thereafter, the cell number was measured using a luminescence measurement instrument (Envision; PerkinElmer Inc.). As a result, it was observed that though the BA/F3 cells died, the number of FLAG-KIF5B-RETv1S expression/BA/F3 cells and FLAG-KIF5B-RETv1L expression/BA/F3 cells was increased by 27 times and 26 times respectively compared to the day of seeding. From the above result, it was understood that the KIF5B-RET fusion polynucleotide v1S and KIF5B-RET fusion polynucleotide v1L have a growth factor-independent growth accelerating action.

### Example 4

### Examination of Anchorage-Independent Growth Accelerating Action and Tumorigenicity of KIF5B-RET Fusion Polynucleotide v1

In order to subclone the full length ORF of KIF5B-RETv1S into a lentiviral expression vector, the primer of SEQ ID NO: 11 into which an SpeI site was introduced and the primer of SEQ ID NO: 12 into which an SalI site was introduced were designed.

By using the above FLAG-KIF5B-RETv1S/pTracer-CMV-bsd as a template, PCR was performed (98° C. for 10 seconds, and then 60° C. for 15 seconds and 68° C. for 3.5 minutes, 35 cycles) using PrimeSTAR HS DNA Polymerase (TAKARA BIO INC.), thereby obtaining PCR products of about 3 kb. The PCR products were purified (QIAquick Gel Extraction Kit; QIAGEN), and products obtained when the PCR products were digested with SpeI and SalI were cloned into SpeI-XhoI site present in multicloning site of the lentiviral expression vector (pLenti 6.3/V5-TOPO; Life Technologies) (the resultant was named FLAG-KIF5B-RETv1S/pLenti 6.3).

3 μg of the FLAG-KIF5B-RETv1S/pLenti 6.3 and 9 μg of the packaging plasmid (ViraPower™ Packaging Mix; Life Technologies) were introduced into 293FT cell packaging cells (Life Technologies) by using the transfection reagent (LipofectAmine 2000; Life Technologies). After 3 days, the culture supernatant was collected as lentivirus, Polybrene (Sigma-Aldrich Co. LLC.) was added thereto at a concentration of 6 μg/ml, and then mouse NIH3T3 cells were added thereto. After 2 days, the culture supernatant of the NIH3T3 cells was replaced to medium obtained by adding 10% bovine serum (Invitrogen) and 5 μg/ml of Blastcidin (Invivogen) to DMEM medium (Invitrogen), and the cells were continuously cultured for 2 more weeks. As a result, NIH3T3 cells (named FLAG-KIF5B-RETv1S/NIH3T3) stably expressing KIF5B-RETv1S in which FLAG was added to the N-terminal were obtained.

The FLAG-KIF5B-RETv1S/NIH3T3 cells and the NIH3T3 cells as the parent strain were seeded respectively into a medium containing 10% fetal bovine serum at 3×10³ cells per well in a 96-well spheroid plate (Sumiron Celltight Spheriod; SUMITOMO BAKELITE CO., LTD.). After the cells were cultured at 37° C. in the presence of 5% CO₂, the number of cells obtained the next day (Day 1) and after 6 days (Day 6) was measured according to the method described in the manual of cell counting reagent (CellTiter-Glo™ Luminescent Cell Viability Assay; Promega Corporation). For detection, a luminescence measurement instrument (Envision; PerkinElmer Inc.) was used. It was confirmed that while the cell count of the NIH3T3 cells did not increase from Day 1 to Day 6, the cell count of the FLAG-KIF5B-RETv1S/NIH3T3 cells increased from Day 1 to Day 6. The result clearly showed that the growth of FLAG-KIF5B-RETv1S/NIH3T3 cells is anchorage-independent cell growth.

Subsequently, in order to examine in-vivo tumorigenicity, the present cell lines were transplanted into nude mice to examine tumorigenicity. The FLAG-KIF5B-RETv1S/NIH3T3 cells were subcutaneously inoculated into nude mice at 3×10⁶ cells, and the mice were observed for 8 days. As a result, the tumor formation was confirmed. This result showed that the KIF5B-RET fusion polynucleotide v1S is a gene responsible for cancer.

### Example 5

### Detection of KIF5B-RET Fusion Polypeptide v1S

A method of detecting the KIF5B-RET fusion polypeptide v1S in cells was established as follows. The FLAG-KIF5B-RETv1S/NIH3T3 cells were lysed in the cell lysis solution (50 mM Hepes pH 7.0, 150 mM NaCl, 1 mM EDTA, 2.5 mM EGTA, 10% glycerol, 0.1% Tween20, protease inhibitor (complete protease inhibitor cocktail; Roche Inc.)) and treated with sonication, followed by centrifugation. Thereafter, protein G beads (Protein G Sepharose 4 Fast Flow; GE Healthcare Inc.) and an anti-RET antibody (Cell Signaling Technology Inc.) or rabbit IgG (Cell Signaling Technology Inc.) were added to the obtained supernatant protein, followed by immunoprecipitation overnight at 4° C. After centrifugation, the precipitate was washed five times with the cell lysis solution and suspended in SDS lysis solution. Immunoblotting was performed on the supernatant thereof by using anti-KIF5B antibody (Sigma-Aldrich Co. LLC.). As a result, though detected in the product of immunoprecipitation performed using the anti-RET antibody, the KIF5B-RET fusion polypeptide v1S was not detected in the product of immunoprecipitation performed using the rabbit IgG This result clearly showed that combining the anti-RET antibody with the anti-KIF5B antibody makes it possible to detect the presence of the KIF5B-RET fusion polypeptide v1S in cancer cells or cancer tissues expressing the KIF5B-RET fusion polypeptide v1S, and to determine cancer patients positive for KIF5B-RET fusion polypeptide v1S.

### Example 6

### Anchorage-Independent Cell Growth Inhibitory Action of KIF5B-RET Fusion Polypeptide Inhibitor Against KIF5B-RET Fusion Polypeptide v1S-Expressing 3T3 Cell

The measurement (colony method or the like) of anchorage-independent cell growth is known to be a system for examining the anticancer action (pharmacological effect) of a compound (“Clinical Oncology” 2^{nd} edition, Japanese Journal of Cancer & Chemotherapy). As a method that measures the growth of non-adherent cells unlike the colony method, there is a method using a spheroid plate as described above.

The FLAG-KIF5B-RETv1S/NIH3T3 cells were seeded at 2×10³ cells per well in a 96-well spheroid plate (Sumiron Celltight Spheriod; SUMITOMO BAKELITE CO., LTD.) by using a DMEM medium containing 10% fetal bovine serum. In addition, a plate to which only a medium was added was prepared as a positive control. After the cells were cultured overnight at 37° C. in the presence of 5% CO₂, anti-cancer drug Sunitinib, Sorafenib or Vandetanib was added thereto at a final concentration of 1.1 μM. As a negative control, DMSO as solvent of the compound was added thereto at the same concentration (0.1%) as the compound. Thereafter, the cells were cultured for 5 days at 37° C. in the presence of 5% CO₂, and the cell counting reagent (CellTiter-Gio™ Luminescent Cell Viability Assay; Promega Corporation) was added thereto, followed by stirring for 20 minutes. Thereafter, the cells were measured using a luminescence measurement instrument (Envision; PerkinElmer Inc.). The values of positive control and negative control were regarded as 100% inhibition value and 0% inhibition value respectively to calculate the growth inhibition rate (%) of the respective compounds. As a result, the inhibition rates (%) of Sunitinib, Sorafenib and Vandetanib were 77%, 96% and 83% respectively.

The inhibitory effect of compounds against phosphorylation of KIF5B-RET in the FLAG-KIF5B-RETv1S/NIH3T3 cells was tested.

The FLAG-KIF5B-RETv1S/NIH3T3 cells were seeded at 2×10⁵ cells, and on the next day, Sunitinib, Sorafenib and Vandetanib were added thereto respectively at a concentration of 1 μM, and the resultant was cultured for 4 hours. Thereafter, the cells were lysed with a cell lysis solution (50 mM Tris.HCl (pH 7.4), 150 mM NaCl, 1% Triton X100, 5 mM EDTA, 5 mM EGTA, 1 mM NaVO₄, 1 mM DTT, a protease inhibitor cocktail complete). An SDS lysis solution was added thereto, immunoblotting was performed using anti-phosphorylated RET antibody (SANTA-CRUZ) specifically recognizing RET in which a 1062^{nd} tyrosine residue was phosphorylated and anti-RET antibody (Cell Signaling Technology, Inc.), and the phosphorylated amount and the total protein amount of about 110 Da of the phosphorylataed KIF5B-RET fusion polypeptide v1S were quantitatively calculated using the image analysis system (VersaDoc Imaging System; Bio-Rad Laboratories, Inc.). A value obtained by dividing the phosphorylated amount by the total protein amount was calculated, and an inhibition rate at the time when the compound was not added (when DMSO as a solvent of the compound was added) were taken as a control (0% inhibition rate) to calculate a phosphorylation inhibition rate at the time when the respective compound was added at 1 μM. As a result, the inhibition rates of Sunitinib, Sorafenib and Vandetanib were 57%, 94% and 74% respectively. From the results, it was understood that Sunitinib, Sorafenib and Vandetanib are KIF5B-RET fusion polypeptide v1S inhibitors.

The above results show that the KIF5B-RET fusion polypeptide inhibitor can inhibit the growth of cancer cells and tumors expressing the KIF5B-RET fusion polypeptide v1S.

It was demonstrated that if subjects of treatment using a KIF5B-RET fusion polypeptide inhibitor, for whom the treatment is expected to be effective, is screened using the detection method of the present invention, tailor-made treatment can be provided.

### INDUSTRIAL APPLICABILITY

The detection method of the present invention is useful for detecting cancer patients positive for the fusion protein of the present specification. The detection kit and the primer set of the present invention can be used for the detection method.

### SEQUENCE LISTING FREE TEXT

The numerical index <223> of the following sequence listing describes “Artificial Sequence”. Specifically, the respective base sequences represented by SEQ ID NOs: 5 to 8, 11 and 12 of the sequence listing are primer sequences synthesized artificially.

1. A method of detecting a fusion gene of a KIF5B gene and a RET gene, or a method of detecting a fusion protein encoded by a fusion gene of a KIF5B gene and a RET gene, the method comprising:
of detecting, in a sample obtained from a test subject, the presence of a polynucleotide encoding a tumorigenic polypeptide comprising an amino acid sequence having 90% or higher identities with the amino acid sequence of SEQ ID NO: 2 or 4 or
detecting, in a sample obtained from a test subject, the presence of a tumorigenic polypeptide comprising an amino acid sequence having 90% or higher identities with the amino acid sequence of SEQ ID NO: 2 or 4.

2. The method according to claim 1, wherein the tumorigenic polypeptide comprises either:
the amino acid sequence of SEQ ID NO: 2 or 4, or
an amino acid sequence obtained by a process comprising deletion, substitution, insertion, or any combination thereof, of from 1 to 10 amino acids in the amino acid sequence of SEQ ID NO: 2 or 4.

3. The method according to claim 1,
wherein the polypeptide consists of the amino acid sequence of SEQ ID NO: 2 or 4.

4. A kit for detecting a fusion gene of a KIF5B gene and a RET gene, the kit comprising:
a sense primer and an antisense primer capable of specifically amplifying a polynucleotide encoding a tumorigenic polypeptide comprising an amino acid sequence having 90% or higher identities with the amino acid sequence of SEQ ID NO: 2 or 4.

5. The kit according to claim 4, wherein the tumorigenic polypeptide comprises either:
the amino acid sequence of SEQ ID NO: 2 or 4, or
an amino acid sequence obtained by a process comprising deletion, substitution, insertion, or a combination thereof, of from 1 to 10 amino acids in the amino acid sequence of SEQ ID NO: 2 or 4.

6. The kit according to claim 4,
wherein the polypeptide consists of the amino acid sequence of SEQ ID NO: 2 or 4.

7. A primer set for detecting a fusion gene of a KIF5B gene and a RET gene, the set comprising:
a sense primer capable of amplifying a portion encoding the KIF5B gene; and
an antisense primer capable of amplifying a portion encoding the RET gene,
wherein the antisense primer consists of a nucleic acid molecule that is hybridized with a polynucleotide encoding a tumorigenic polypeptide comprising an amino acid sequence having 90% or higher identities with the amino acid sequence of SEQ ID NO: 2 or 4 under a stringent condition, and
the sense primer consists of a nucleic acid molecule that is hybridized with a complementary strand of the polynucleotide under a stringent condition.

8. A primer set, comprising:
a sense primer consisting of an oligonucleotide with at least any 16 consecutive bases located at base numbers 1 and 1725 of SEQ ID NO: 1 and an antisense primer consisting of an oligonucleotide complementary to an oligonucleotide with at least any 16 consecutive bases located at base numbers 1726 and 2808 of SEQ ID NO: 1, or
complementary strands thereof,
wherein a size of an amplification product amplified by the sense primer and the antisense primer is 1 kb or less.

9. A primer set, comprising:
a sense primer consisting of an oligonucleotide with at least any 16 consecutive bases located at base numbers 1 and 1725 of SEQ ID NO: 3 and an antisense primer consisting of an oligonucleotide complementary to an oligonucleotide with at least any 16 consecutive bases located at base numbers 1726 and 2934 of SEQ ID NO: 3, or
complementary strands thereof,
wherein a size of an amplification product amplified by the sense primer and the antisense primer is 1 kb or less.

10. A method of detecting the presence of cancer positive for a fusion gene having RET kinase domain, the method comprising:
detecting, in a sample obtained from a test subject, the presence of a polynucleotide that encodes a tumorigenic polypeptide comprising an amino acid sequence having 90% or higher identities with the amino acid sequence of SEQ ID NO: 2 or 4.

11. The method according to claim 10, wherein the tumorigenic polypeptide comprises either:
the amino acid sequence of SEQ ID NO: 2 or 4, or
an amino acid sequence obtained by a process comprising deletion, substitution, insertion, or any combination thereof, of from 1 to 10 amino acids in the amino acid sequence of SEQ ID NO: 2 or 4.

12. The method according to claim 10, wherein the polypeptide is a polypeptide consisting of the amino acid sequence of SEQ ID NO: 2 or 4.

13. The method of claim 10, wherein the cancer is lung cancer.

14. The set of claim 7, wherein the tumorigenic polypeptide comprises either:
the amino acid sequence of SEQ ID NO: 2 or 4, or
an amino acid sequence obtained by a process comprising deletion, substitution, insertion, or any combination thereof, of from 1 to 10 amino acids in the amino acid sequence of SEQ ID NO: 2 or 4.

15. The set of claim 7, wherein the polypeptide consists of the amino acid sequence of SEQ ID NO: 2 or 4.


SEQUENCE LISTING (12 sequences)

SEQ ID NO: 1 - DNA, 2808 bp, Homo sapiens
  CDS (1)..(2808)
atggcggacctggccgagtgcaacatcaaagtgatgtgtcgcttcagacctctcaacgagtctgaagtgaaccgcggcgacaagtacatcgccaagtttcagggagaagacacggtcgtgatcgcgtccaagccttatgcatttgatcgggtgttccagtcaagcacatctcaagagcaagtgtataatgactgtgcaaagaagattgttaaagatgtacttgaaggatataatggaacaatatttgcatatggacaaacatcctctgggaagacacacacaatggagggtaaacttcatgatccagaaggcatgggaattattccaagaatagtgcaagatatttttaattatatttactccatggatgaaaatttggaatttcatattaaggtttcatattttgaaatatatttggataagataagggacctgttagatgtttcaaagaccaacctttcagttcatgaagacaaaaaccgagttccctatgtaaaggggtgcacagagcgttttgtatgtagtccagatgaagttatggataccatagatgaaggaaaatccaacagacatgtagcagttacaaatatgaatgaacatagctctaggagtcacagtatatttcttattaatgtcaaacaagagaacacacaaacggaacaaaagctgagtggaaaactttatctggttgatttagctggtagtgaaaaggttagtaaaactggagctgaaggtgctgtgctggatgaagctaaaaacatcaacaagtcactttctgctcttggaaatgttatttctgctttggctgagggtagtacatatgttccatatcgagatagtaaaatgacaagaatccttcaagattcattaggtggcaactgtagaaccactattgtaatttgttgctctccatcatcatacaatgagtctgaaacaaaatctacactcttatttggccaaagggccaaaacaattaagaacacagtttgtgtcaatgtggagttaactgcagaacagtggaaaaagaagtatgaaaaagaaaaagaaaaaaataagatcctgcggaacactattcagtggcttgaaaatgagctcaacagatggcgtaatggggagacggtgcctattgatgaacagtttgacaaagagaaagccaacttggaagctttcacagtggataaagatattactcttaccaatgataaaccagcaaccgcaattggagttataggaaattttactgatgctgaaagaagaaagtgtgaagaagaaattgctaaattatacaaacagcttgatgacaaggatgaagaaattaaccagcaaagtcaactggtagagaaactgaagacgcaaatgttggatcaggaggagcttttggcatctaccagaagggatcaagacaatatgcaagctgagctgaatcgccttcaagcagaaaatgatgcctctaaagaagaagtgaaagaagttttacaggccctagaagaacttgctgtcaattatgatcagaagtctcaggaagttgaagacaaaactaaggaatatgaattgcttagtgatgaattgaatcagaaatcggcaactttagcgagtatagatgctgagcttcagaaacttaaggaaatgaccaaccaccagaaaaaacgagcagctgagatgatggcatctttactaaaagaccttgcagaaataggaattgctgtgggaaataatgatgtaaaggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagcttcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 2 - protein, 935 aa, Homo sapiens
MADLAECNIKVMCRFRPLNESEVNRGDKYIAKFQGEDTVVIASKPYAFDRVFQSSTSQEQVYNDCAKKIVKDVLEGYNGTIFAYGQTSSGKTHTMEGKLHDPEGMGIIPRIVQDIFNYIYSMDENLEFHIKVSYFEIYLDKIRDLLDVSKTNLSVHEDKNRVPYVKGCTERFVCSPDEVMDTIDEGKSNRHVAVTNMNEHSSRSHSIFLINVKQENTQTEQKLSGKLYLVDLAGSEKVSKTGAEGAVLDEAKNINKSLSALGNVISALAEGSTYVPYRDSKMTRILQDSLGGNCRTTIVICCSPSSYNESETKSTLLFGQRAKTIKNTVCVNVELTAEQWKKKYEKEKEKNKILRNTIQWLENELNRWRNGETVPIDEQFDKEKANLEAFTVDKDITLTNDKPATAIGVIGNFTDAERRKCEEEIAKLYKQLDDKDEEINQQSQLVEKLKTQMLDQEELLASTRRDQDNMQAELNRLQAENDASKEEVKEVLQALEELAVNYDQKSQEVEDKTKEYELLSDELNQKSATLASIDAELQKLKEMTNHQKKRAAEMMASLLKDLAEIGIAVGNNDVKEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 3 - DNA, 2934 bp, Homo sapiens
  CDS (1)..(2934)
atggcggacctggccgagtgcaacatcaaagtgatgtgtcgcttcagacctctcaacgagtctgaagtgaaccgcggcgacaagtacatcgccaagtttcagggagaagacacggtcgtgatcgcgtccaagccttatgcatttgatcgggtgttccagtcaagcacatctcaagagcaagtgtataatgactgtgcaaagaagattgttaaagatgtacttgaaggatataatggaacaatatttgcatatggacaaacatcctctgggaagacacacacaatggagggtaaacttcatgatccagaaggcatgggaattattccaagaatagtgcaagatatttttaattatatttactccatggatgaaaatttggaatttcatattaaggtttcatattttgaaatatatttggataagataagggacctgttagatgtttcaaagaccaacctttcagttcatgaagacaaaaaccgagttccctatgtaaaggggtgcacagagcgttttgtatgtagtccagatgaagttatggataccatagatgaaggaaaatccaacagacatgtagcagttacaaatatgaatgaacatagctctaggagtcacagtatatttcttattaatgtcaaacaagagaacacacaaacggaacaaaagctgagtggaaaactttatctggttgatttagctggtagtgaaaaggttagtaaaactggagctgaaggtgctgtgctggatgaagctaaaaacatcaacaagtcactttctgctcttggaaatgttatttctgctttggctgagggtagtacatatgttccatatcgagatagtaaaatgacaagaatccttcaagattcattaggtggcaactgtagaaccactattgtaatttgctgctctccatcatcatacaatgagtctgaaacaaaatctacactcttatttggccaaagggccaaaacaattaagaacacagtttgtgtcaatgtggagttaactgcagaacagtggaaaaagaagtatgaaaaagaaaaagaaaaaaataagatcctgcggaacactattcagtggcttgaaaatgagctcaacagatggcgtaatggggagacggtgcctattgatgaacagtttgacaaagagaaagccaacttggaagctttcacagtggataaagatattactcttaccaatgataaaccagcaaccgcaattggagttataggaaattttactgatgctgaaagaagaaagtgtgaagaagaaattgctaaattatacaaacagcttgatgacaaggatgaagaaattaaccagcaaagtcaactggtagagaaactgaagacgcaaatgttggatcaggaggagcttttggcatctaccagaagggatcaagacaatatgcaagctgagctgaatcgccttcaagcagaaaatgatgcctctaaagaagaagtgaaagaagttttacaggccctagaagaacttgctgtcaattatgatcagaagtctcaggaagttgaagacaaaactaaggaatatgaattgcttagtgatgaattgaatcagaaatcggcaactttagcgagtatagatgctgagcttcagaaacttaaggaaatgaccaaccaccagaaaaaacgagcagctgagatgatggcatctttactaaaagaccttgcagaaataggaattgctgtgggaaataatgatgtaaaggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagcttcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcgtacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggcatgtcagacccgaactggcctggagagagtcctgtaccactcacgagagctgatggcactaacactgggtttccaagatatccaaatgatagtgtatatgctaactggatgctttcaccctcagcggcaaaattaatggacacgtttgatagttaa

SEQ ID NO: 4 - protein, 977 aa, Homo sapiens
MADLAECNIKVMCRFRPLNESEVNRGDKYIAKFQGEDTVVIASKPYAFDRVFQSSTSQEQVYNDCAKKIVKDVLEGYNGTIFAYGQTSSGKTHTMEGKLHDPEGMGIIPRIVQDIFNYIYSMDENLEFHIKVSYFEIYLDKIRDLLDVSKTNLSVHEDKNRVPYVKGCTERFVCSPDEVMDTIDEGKSNRHVAVTNMNEHSSRSHSIFLINVKQENTQTEQKLSGKLYLVDLAGSEKVSKTGAEGAVLDEAKNINKSLSALGNVISALAEGSTYVPYRDSKMTRILQDSLGGNCRTTIVICCSPSSYNESETKSTLLFGQRAKTIKNTVCVNVELTAEQWKKKYEKEKEKNKILRNTIQWLENELNRWRNGETVPIDEQFDKEKANLEAFTVDKDITLTNDKPATAIGVIGNFTDAERRKCEEEIAKLYKQLDDKDEEINQQSQLVEKLKTQMLDQEELLASTRRDQDNMQAELNRLQAENDASKEEVKEVLQALEELAVNYDQKSQEVEDKTKEYELLSDELNQKSATLASIDAELQKLKEMTNHQKKRAAEMMASLLKDLAEIGIAVGNNDVKEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGMSDPNWPGESPVPLTRADGTNTGFPRYPNDSVYANWMLSPSAAKLMDTFDS

SEQ ID NO: 5 - DNA, 35 bp, Artificial
  Description of Artificial Sequencean artificially systhesized primer sequence
gcggccgcaagatggcggacctggccgagtgcaac

SEQ ID NO: 6 - DNA, 35 bp, Artificial
  Description of Artificial Sequencean artificially systhesized primer sequence
gcggccgcctagaatctagtaaatgcatgggaaat

SEQ ID NO: 7 - DNA, 35 bp, Artificial
  Description of Artificial Sequencean artificially systhesized primer sequence
gcggccgcttaactatcaaacgtgtccattaattt

SEQ ID NO: 8 - DNA, 71 bp, Artificial
  Description of Artificial Sequencean artificially systhesized primer sequence
gtggcggccgcaccatggactacaaagacgatgacgacaagcttgcggacctggccgagtgcaacatcaaa

SEQ ID NO: 9 - DNA, 25 bp, Homo sapiens
aaagaccttgcagaaataggaattg

SEQ ID NO: 10 - DNA, 22 bp, Homo sapiens
gaattcccactttggatcctcc

SEQ ID NO: 11 - DNA, 36 bp, Artificial
  Description of Artificial Sequencean artificially systhesized primer sequence
caccactagtatggactacaaagacgatgacgacaa

SEQ ID NO: 12 - DNA, 39 bp, Artificial
  Description of Artificial Sequencean artificially systhesized primer sequence
aatgtcgacctagaatctagtaaatgcatgggaaattct